(19) United States
(12) Patent Application Publication
(10) Pub. No.: US 2007/0203203 A1
(43) Pub. Date: Aug. 30, 2007

(54) Composition and Method for Treating Fibrotic Diseases

(76) Inventors: Li Jian Tao, Changsha (CN); Gao Yun Hu, Changsha (CN); Yuenian Eric Shi, Roslyn Heights, NY (US)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/742,664

(22) Filed: May 1, 2007

Related U.S. Application Data
(60) Provisional application No. 60/849,039, filed on Oct. 3, 2006.

(30) Foreign Application Priority Data
Apr. 11, 2006 (CN) PCT/CN06/00651
Apr. 13, 2005 (CN) 200510031445.7

(51) Int. Cl. A61K 31/4412

(52) U.S. Cl. 514/345

(57) ABSTRACT

The present invention discloses 5-methyl-1-(substituted phenyl)-2(1H)-pyridones have enhanced anti-fibrotic activities than 5-methyl-1-(non-substituted phenyl)-2(1H)-pyridones. An representative example of 5-methyl-(1-substituted phenyl)-2(1H)-pyridones is 1-(3′-fluorophenyl)-5-methyl-2(1H)-pyridone. Accordingly, there are provided compositions comprising one or more compounds selected from the group consisting of 5-methyl-1-(substituted phenyl)-2(1H)-pyridones and methods of using the same to treat or prevent fibrosis diseases.

### CROSS-REFERENCE TO RELATED APPLICATIONS

This application claims the benefit of priority of U.S. Ser. No. 60/849,039, filed Oct. 3, 2006, International Application No. PCT/CN2006/000651, filed Apr. 11, 2006, and Chinese application No. 200510031445.7, filed Apr. 13, 2005. The entire contents and disclosures of the preceding applications are incorporated by reference into this application.

Throughout this application, various references or publications are cited. Disclosures of these references or publications in their entireties are hereby incorporated by reference into this application in order to more fully describe the state of the art to which this invention pertains.

### FIELD OF THE INVENTION

This invention is related to compositions comprising 5-methyl-1-(substituted phenyl)-2(1H)-pyridones and methods of using the same to treat fibrotic diseases.

### BACKGROUND OF THE INVENTION

Fibrosis can occur in various organs or tissues, causing reduction of healthy cells within any organ or tissue and increase in the mass of fibrotic connective tissues, eventually damage the normal structure of the organs or tissues. The damages can impair the physiological and biochemical functions of the affected organs or tissues, and may cause organ shut down completely. The pathogenesis, diagnostic methods, methods of prevention and treatment for organ and tissue fibrosis have been studied extensively. Much progress has been made in certain areas. However there are still many challenges, especially in the area of developing effective therapeutics.

It is generally believed that fibrosis of organs or tissues are caused by multiple factors such as inflammation, immunological reactions, ischemia, hemodynamics change etc. that cause inflammatory denaturing and narcosis of parenchymal cells. The impaired parenchymal cells in turn activate macrophages to release numerous cytokines and growth factors, among which TGF-β is a critical one. TGF-β can activate quiescent extracellular matrix (ECM) producing cells and turn them into myofibroblast. The newly formed fibroblasts not only increase production of collagen, a key protein of ECM, but also decrease destruction of ECM. The net result is accumulation of extracellular matrix that leads to organ or tissue fibrosis. Thus, initiation and development of organ or tissue fibrosis is the results of inflammatory response and production of inflammatory cytokines, mainly TGF-β. Logically, due to the crucial role of TGF-β on ECM accumulation and formation of organ or tissue fibrosis, one of the important goals in early development or screening of antifibrotic drugs is to find a way to inhibit the production of pro-inflammatory cytokines, e.g., TGF-β. However, more convincing data for any antifibrotic drug candidate will obviously come from the test using various in vivo fibrotic models.

A number of compounds that exhibit anti-inflammatory and anti-fibrotic activities have been described. U.S. Pat. Nos. 3,839,346, 3,974,281, 4,042,699 and 4,052,509 published a total of 29 compounds with the following pyridone-like formula (I): 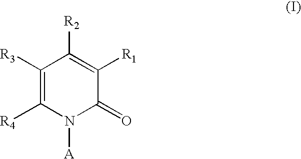
wherein A is an aromatic group. These compounds have good anti-inflammatory and analgesic activities and can reduce serum levels of uric acid and glucose. One compound in particular, 5-methyl-1-phenyl-2(1H)-pyridone, has the best activity and low toxicity.

U.S. Pat. No. 5,310,562 reported anti-fibrotic activity for 5-methyl-1-phenyl-2(1H)-pyridone (PIRFENIDONE, PFD). U.S. Pat. Nos. 5,518,729 and 5,716,632 described the anti-fibrotic activities of additional 44 compounds of either N-substituted 2(1H)-pyridone (I) or N-substituted 3(1H)-pyridone.

The efficacy of anti-fibrotic activity of 5-methyl-1-phenyl-2(1H)-pyridone (PIRFENIDONE, PFD) has been further demonstrated in various animal models and human clinical trials (Shimizu et al., Pirfenidone prevents collagen accumulation in the remnant kidney in rats with partial nephrectomy. Kidney Int. 52(Suppl 63):S239-243 (1997); Raghu et al., Treatment of idiopathic pulmonary fibrosis with a new antifibrotic agent, pirfenidone. Am. J. Respir. Crit. Care Med. 159:1061-1069 (1999)). These studies indicated that PIRFENIDONE not only prevents but also reverses the accumulation of excess extracellular matrix. The pharmacological mechanism of PIRFENIDONE has not been fully understood yet, but data to date indicate that PIRFENIDONE is an effective compound to down-regulate cytokines (including TGF-β), and decrease the activity of fibroblasts through regulating multiple factors.

A Chinese patent ZL02114190.8 described the identification and synthesis of total 38 new 5-methyl-1-(substituted phenyl)-2(1H)-pyridone compounds, having the following general structural formula (II):

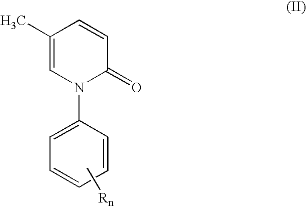

U.S. Pat. No. 5,716,632 listed 6 structural formulas of 5-methyl-1-(substituted phenyl)-2(1H)-pyridone originally described by Gadekar in U.S. Pat. No. 3,974,281. For these “substituted phenyl” compounds, Gadekar established a structure-activity relationship that teaches non-substituted phenyl as the compound with the best biological activities. However, it has been reported that a 5-methyl-1-(substituted phenyl)-2(1H)-pyridone, 1-(3′-fluorophenyl)-5-methyl-2(1H)-pyridone, displayed certain biological activities in vitro (J. Cent. South Univ. Med. Sci. 29:139 (2004)). Thus, it is of interest to determine whether substituted phenyl compounds having structural formula II would have any desirable anti-fibrotic activity.

### SUMMARY OF THE INVENTION

The present invention discloses a composition for treating fibrotic diseases comprising novel compounds in the 5-methyl-1-(substituted phenyl)-2(1H)-pyridone family. The efficacy of a representative substituted phenyl pyridone, 1-(3′-fluorophenyl)-5-methyl-2(1H)-pyridone (AKF-PD), was demonstrated in various animal models of fibrosis diseases. AKF-PD was synthesized according to a process similar to the one described in Chinese patent ZL02114190.8, the disclosure of which is incorporated herein by reference. As compared with PIRFENIDONE (PFD), a leading experimental drug in the field, AKF-PD has better anti-fibrotic activities but with much less toxicity. The results presented herein demonstrate that 5-methyl-1-(substituted phenyl)-2(1H)-pyridones can be used as more potent anti-fibrotic drugs for organ or tissue fibrosis with much less toxic effect.

In one embodiment, the present invention provides a composition comprising one or more 5-methyl-1-(substituted phenyl)-2(1H)-pyridone in an amount effective for treating organ or tissue fibrosis, said 5-methyl-1-(substituted phenyl)-2(1H)-pyridone having a general structural formula of: 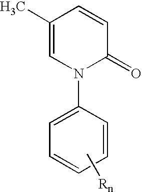
wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

The present invention also provides a group of novel compounds of 5-methyl-1-(substituted phenyl)-2(1H)-pyridones.

The present invention also provides methods of using a composition comprising one or more 5-methyl-1-(substituted phenyl)-2(1H)-pyridone as disclosed herein for treating organ or tissue fibrosis.

### BRIEF DESCRIPTION OF THE DRAWINGS

These and other features and advantages of this invention will be evident from the following detailed description of preferred embodiments when read in conjunction with the accompanying drawings in which:

FIG. 1 shows the inhibition of TGF-β production in rat UUO model (Unilateral Ureteral Obstruction) by 1-(3′-fluorophenyl)-5-methyl-2(1H)-pyridone (AKF-PD). A: Control, sham operation; B: Disease model, UUO; C: Disease model+AKF-PD. Brown or dark color indicate TGF-β positive cells.

FIG. 2 shows the inhibition of type I & III collagen accumulation in rat UUO model (Unilateral Ureteral Obstruction) by AKF-PD. Section A-C: type I collagen; section D-F: type III collagen. A and D: control rat with sham operation; B and E: disease model with UUO; C and F, disease model+AKF-PD.

FIG. 3 shows the suppression of Schistosome-induced liver fibrotic nodule by AKF-PD. A: Control rat; B: Fibrotic rat (Schistosoma-induced liver fibrosis); C: Fibrotic rat+Pyquiton; D: Fibrotic rat+Interferon-γ; E: Fibrotic rat+AKF-PD. Brown or dark color indicate fibrotic nodule.

FIG. 4 shows reduction of type I collagen accumulation in Schistosome-induced liver fibrotic nodule by AKF-PD. A: Control rat; B: Fibrotic rat (Schistosoma-induced liver fibrosis); C: Fibrotic rat+Pyquiton; D: Fibrotic rat+Interferon-γ; E: Fibrotic rat+AKF-PD. Brown or dark color indicate collagen I staining.

### DETAILED DESCRIPTION OF THE INVENTION

In the present invention, the term “anti-organ or tissue fibrosis” means preventing fibrosis in organs or tissues, slowing or stopping fibrosis process in organs or tissues, and/or reversing the fibrotic lesion in organs or tissues.

It was shown previously that 5-methyl-1-phenyl-2(1H)-pyridone (PIRFENIDONE) with non-substituted phenyl is a compound with desirable anti-fibrotic activities, which is consistent with Gadekar's structure-activity relationship: non-substituted phenyl is better than substituted phenyl. Thus, it is unexpected to find 5-methyl-1-(substituted phenyl)-2(1H)-pyridones (structural formula II) exhibiting more potent anti-fibrotic activities and much less toxicity than 5-methyl-1-phenyl-2(1H)-pyridone (PIRFENIDONE) as shown herein.

The present invention discloses a new structure-activity relationship: 5-methyl-1-(substituted phenyl)-2(1H)-pyridones are better than 5-methyl-1-phenyl-2(1H)-pyridone. In structural formula II, when n=1, the following compounds all have activities inhibiting fibroblast: 1-(3′-bromophenyl)-5-methyl-2(1H)-pyridone, 1-(3′-fluorophenyl)-5-methyl-2(1H)-pyridone, 1-(3′-iodophenyl)-5-methyl-2(1H)-pyridone, 5-methyl-1-(3′-methylphenyl)-2(1H)-pyridone. A side-by-side comparison further indicates that the inhibitory activity to fibroblasts is: F>Br>Cl>H.

A representative example of 5-methyl-1-(substituted phenyl)-2(1H)-pyridone is 1-(3′-fluorophenyl)-5-methyl-2(1H)-pyridone. It was determined that the anti-fibrotic activities of 1-(3′-fluorophenyl)-5-methyl-2(1H)-pyridone are enhanced compared to PIRFENIDONE. Moreover, the toxicity of 5-methyl-1-(substituted phenyl)-2(1H)-pyridone is drastically decreased. As presented herein, the LD₅₀ for 1-(3′-fluorophenyl)-5-methyl-2(1H)-pyridone is only 30% percent of that of PIRFENIDONE. It is important to note that as all fibrotic diseases are chronic illness, it is expected that any reparation or prevention is going to be a lengthy process, which warrants a long-term pharmacological intervention. Therefore, just like any long-term drug use, it is very desirable to have anti-fibrotic drugs with low toxicity.

Other examples of 5-methyl-1-(substituted phenyl)-2(1H)-pyridone (structural formula II) include, but are not limited to, the following compounds:

When n=1 and R═Br, the compounds can be 1-(2′-bromophenyl)-5-methyl-2(1H)-pyridone, 1-(3′-bromophenyl)-5-methyl-2(1H)-pyridone, or 1-(4′-bromophenyl)-5-methyl-2(1H)-pyridone.

When n=1 and R═F, the compounds can be 1-(2′-fluorophenyl)-5-methyl-2(1H)-pyridone, 1-(3′-fluorophenyl)-5-methyl-2(1H)-pyridone, or 1-(4′-fluorophenyl)-5-methyl-2(1H)-pyridone.

When n=1 and R═I, the compounds can be 1-(2′-iodophenyl)-5-methyl-2(1H)-pyridone, 1-(3′-iodophenyl)-5-methyl-2(1H)-pyridone, or 1-(4′-iodophenyl)-5-methyl-2(1H)-pyridone.

When n=2 and R═F, Br, or Cl, the compounds can be 1-(2′,3′-dibromophenyl)-5-methyl-2(1H)-pyridone, 1-(2′,4′-dibromophenyl)-5-methyl-2(1H)-pyridone, 1-(2′,5′-dibromophenyl)-5-methyl-2(1H)-pyridone, 1-(2′,6′-dibromophenyl)-5-methyl-2(1H)-pyridone, 1-(3′,4′-dibromophenyl)-5-methyl-2(1H)-pyridone, 1-(3′,5′-dibromophenyl)-5-methyl-2(1H)-pyridone, 1-(2′,3′-dichlorophenyl)-5-methyl-2(1H)-pyridone, 1-(2′,4′-dichlorophenyl)-5-methyl-2(1H)-pyridone, 1-(2′,5′-dichlorophenyl)-5-methyl-2(1H)-pyridone, 1-(2′,6′-dichlorophenyl)-5-methyl-2(1H)-pyridone, 1-(3′,4′-dichlorophenyl)-5-methyl-2(1H)-pyridone, 1-(3′,5′-dichlorophenyl)-5-methyl-2(1H)-pyridone, 1-(2′,3′-difluorophenyl)-5-methyl-2(1H)-pyridone, 1-(2′,4′-difluorophenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluorophenyl)-5-methyl-2(1H)-pyridone, or 1-(3′,5′-difluorophenyl)-5-methyl-2(1H)-pyridone.

When n=1 or 2 and R=trifluoromethyl, the compounds can be 5-methyl-1-(2′-trifluoromethylphenyl)-2(1H)-pyridone, 5-methyl-1-(4′-trifluoromethylphenyl)-2(1H)-pyridone, 1-(2′,3′-bis-trifluoromethylphenyl)-5-methyl-2(1H)-pyridone, 1-(2′,4′-bis-trifluoromethylphenyl)-5-methyl-2(1H)-pyridone, 1-(2′,5′-bis-trifluoromethylphenyl)-5-methyl-2(1H)-pyridone, 1-(2′,6′-bis-trifluoromethylphenyl)-5-methyl-2(1H)-pyridone, 1-(3′,4′-bis-trifluoromethylphenyl)-5-methyl-2(1H)-pyridone, or 1-(3′,5′-bis-trifluoromethylphenyl)-5-methyl-2(1H)-pyridone.

When n=1 or 2 and R=methyl, the compounds can be 5-methyl-1-(2′-methylphenyl)-2(1H)-pyridone, 5-methyl-1-(3′-methylphenyl)-2(1H)-pyridone, 1-(2′,3′-dimethylphenyl)-5-methyl-2(1H)-pyridone, 1-(2′,4′-dimethylphenyl)-5-methyl-2(1H)-pyridone, 1-(2′,5′-dimethylphenyl)-5-methyl-2(1H)-pyridone, 1-(2′,6′-dimethylphenyl)-5-methyl-2(1H)-pyridone, 1-(3′,4′-dimethylphenyl)-5-methyl-2(1H)-pyridone, or 1-(3′,5′-dimethylphenyl)-5-methyl-2(1H)-pyridone.

When n=1 or 2 and R=methoxy, the compounds can be 1-(2′-methoxyphenyl)-5-methyl-2(1H)-pyridone, 1-(3′-methoxyphenyl)-5-methyl-2(1H)-pyridone, 1-(2′,3′-dimethoxyphenyl)-5-methyl-2(1H)-pyridone, 1-(2′,4′-dimethoxyphenyl)-5-methyl-2(1H)-pyridone, 1-(2′,5′-dimethoxyphenyl)-5-methyl-2(1H)-pyridone, 1-(2′,6′-dimethoxyphenyl)-5-methyl-2(1H)-pyridone, 1-(3′,4′-dimethoxyphenyl)-5-methyl-2(1H)-pyridone, or 1-(3′,5′-dimethoxyphenyl)-5-methyl-2(1H)-pyridone.

The present invention provides a composition comprising one or more 5-methyl-1-(substituted phenyl)-2(1H)-pyridone in an amount effective for treating organ or tissue fibrosis, said 5-methyl-1-(substituted phenyl)-2(1H)-pyridone having a general structural formula of: 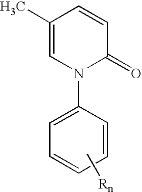
wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

Examples of 5-methyl-1-(substituted phenyl)-2(1H)-pyridones in the above composition include, but are not limited to, 1-(2′-bromophenyl)-5-methyl-2(1H)-pyridone, 1-(3′-bromophenyl)-5-methyl-2(1H)-pyridone, 1-(4′-bromophenyl)-5-methyl-2(1H)-pyridone, 1-(3′-chlorophenyl)-5-methyl-2(1H)-pyridone, 1-(2′-fluorophenyl)-5-methyl-2(1H)-pyridone, 1-(3′-fluorophenyl)-5-methyl-2(1H)-pyridone, 1-(4′-fluorophenyl)-5-methyl-2(1H)-pyridone, 1-(2′-iodophenyl)-5-methyl-2(1H)-pyridone, 1-(3′-iodophenyl)-5-methyl-2(1H)-pyridone, 1-(4′-iodophenyl)-5-methyl-2(1H)-pyridone, 1-(2′,3′-dibromophenyl)-5-methyl-2(1H)-pyridone, 1-(2′,4′-dibromophenyl)-5-methyl-2(1H)-pyridone, 1-(2′,5′-dibromophenyl)-5-methyl-2(1H)-pyridone, 1-(2′,6′-dibromophenyl)-5-methyl-2(1H)-pyridone, 1-(3′,4′-dibromophenyl)-5-methyl-2(1H)-pyridone, 1-(3′,5′-dibromophenyl)-5-methyl-2(1H)-pyridone, 1-(2′,3′-dichlorophenyl)-5-methyl-2(1H)-pyridone, 1-(2′,4′-dichlorophenyl)-5-methyl-2(1H)-pyridone, 1-(2′,5′-dichlorophenyl)-5-methyl-2(1H)-pyridone, 1-(2′,6′-dichlorophenyl)-5-methyl-2(1H)-pyridone, 1-(3′,4′-dichlorophenyl)-5-methyl-2(1H)-pyridone, 1-(3′,5′-dichlorophenyl)-5-methyl-2(1H)-pyridone, 1-(2′,3′-difluorophenyl)-5-methyl-2(1H)-pyridone, 1-(2′,4′-difluorophenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluorophenyl)-5-methyl-2(1H)-pyridone, 1-(3′,5′-difluorophenyl)-5-methyl-2(1H)-pyridone, 5-methyl-1-(2′-trifluoromethylphenyl)-2(1H)-pyridone, 5-methyl-1-(4′-trifluoromethylphenyl)-2(1H)-pyridone, 1-(2′,3′-bis-trifluoromethylphenyl)-5-methyl-2(1H)-pyridone, 1-(2′,4′-bis-trifluoromethylphenyl)-5-methyl-2(1H)-pyridone, 1-(2′,5′-bis-trifluoromethylphenyl)-5-methyl-2(1H)-pyridone, 1-(2′,6′-bis-trifluoromethylphenyl)-5-methyl-2(1H)-pyridone, 1-(3′,4′-bis-trifluoromethylphenyl)-5-methyl-2(1H)-pyridone, or 1-(3′,5′-bis-trifluoromethylphenyl)-5-methyl-2(1H)-pyridone. 5-methyl-1-(2′-methylphenyl)-2(1H)-pyridone, 1-(3′-methylphenyl)-5-methyl-2(1H)-pyridone, 1-(2′,3′-dimethylphenyl)-5-methyl-2(1H)-pyridone, 1-(2′,4′-dimethylphenyl)-5-methyl-2(1H)-pyridone, 1-(2′,5′-dimethylphenyl)-5-methyl-2(1H)-pyridone, 1-(2′,6′-dimethylphenyl)-5-methyl-2(1H)-pyridone, 1-(3′,4′-dimethylphenyl)-5-methyl-2(1H)-pyridone, 1-(3′,5′-dimethylphenyl)-5-methyl-2(1H)-pyridone, 1-(2′-methoxyphenyl)-5-methyl-2(1H)-pyridone, 1-(3′-methoxyphenyl)-5-methyl-2(1H)-pyridone, 1-(2′,3′-dimethoxyphenyl)-5-methyl-2(1H)-pyridone, 1-(2′,4′-dimethoxyphenyl)-5-methyl-2(1H)-pyridone, 1-(2′,5′-dimethoxyphenyl)-5-methyl-2(1H)-pyridone, 1-(2′,6′-dimethoxyphenyl)-5-methyl-2(1H)-pyridone, 1-(3′,4′-dimethoxyphenyl)-5-methyl-2(1H)-pyridone, and 1-(3′,5′-dimethoxyphenyl)-5-methyl-2(1H)-pyridone.

The present invention also provides a compound of 5-methyl-1-(substituted phenyl)-2(1H)-pyridone. Examples of such compounds include, but are not limited to, 1-(2′-fluorophenyl)-5-methyl-2(1H)-pyridone, 1-(3′-fluorophenyl)-5-methyl-2(1H)-pyridone, 1-(4′-fluorophenyl)-5-methyl-2(1H)-pyridone, 1-(2′-iodophenyl)-5-methyl-2(1H)-pyridone, 1-(3′-iodophenyl)-5-methyl-2(1H)-pyridone, 1-(4′-iodophenyl)-5-methyl-2(1H)-pyridone, 1-(2′,3-difluorophenyl)-5-methyl-2(1H)-pyridone, 1-(2′,4′-difluorophenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluorophenyl)-5-methyl-2(1H)-pyridone, 1-(3′,5′-difluorophenyl)-5-methyl-2(1H)-pyridone and 1-(3′,4′-dichlorophenyl)-5-methyl-2(1H)-pyridone.

The present invention also provides a pharmaceutical composition comprising the composition described above and a pharmaceutically acceptable carrier. The pharmaceutical composition can be formulated as solution, tablet, capsule, suppository, inhaler, suspension, gel, cream, or ointment.

The present invention also provides a method of treating organ or tissue fibrosis, comprising the step of administrating a composition comprising one or more compound as described above. The method can be used to treat organ or tissue fibrosis such as glomerulus sclerosis, renal interstitial fibrosis, liver fibrosis, pulmonary fibrosis, peridoneal fibrosis, myocardiac fibrosis, fibrosis of skin, post-surgical adhesion, benign prostatic hypertrophy, musculoskeletal fibrosis, scleroderma, Alzheimer's disease, fibrotic vascular disease, and glaucoma. Examples of amounts effective for treating organ or tissue fibrosis include a daily dosage of about 25 mg to about 6,000 mg, a daily dosage of about 50 mg to about 2000 mg, or a daily dosage of about 100 mg to about 1000 mg. The composition described above can be administered by oral administration, parenteral administration, nasal administration, rectal administration, vaginal administration, ophthalmic application, or topical application.

In one embodiment, the above method comprises administrating 1-(3′-fluorophenyl)-5-methyl-2(1H)-pyridone to a subject in need of such treatment. Subject treated with 1-(3′-fluorophenyl)-5-methyl-2(1H)-pyridone will experience less toxicity than treatment with 5-methyl-1-phenyl-2(1H)-pyridone.

The invention being generally described will be more readily understood by reference to the following examples which are included merely for purposes of illustration of certain aspects and embodiments of the present invention, and are not intended to limit the invention.

In the following examples, myocardiac fibroblasts, skin scar forming fibroblast and human peritoneal mesothelial cells are primary cultures prepared according to common procedure. Other cells are commercially purchased. 1-(3-fluorophenyl)-5-methyl-2(1H)-pyridone (AKF-PD) is prepared as a suspension in 5% carboxymethylcellulose.

### EXAMPLE 1

### Suppression of Mouse Kidney Fibroblast Proliferation by 1-(3′-Fluorophenyl)-5-Methyl-2(1H)-Pyridone and PIRFENIDONE

Cell proliferation was measured by MTT assay. DMEM with 10% fetal serum was used as cell culture medium. The cells were prepared in suspension (1×10⁵/ml), and 100 μL of the suspension was transferred to each well of a 96 wells plate. Once the cells were attached to the plastic, the culture was changed to serum free medium and continued for another 24 hours. The serum free medium was aspirated, and various concentrations of 1-(3′-fluorophenyl)-5-methyl-2(1H)-pyridone (AKF-PD) 1-(3′-bromophenyl)-5-methyl-2(1H)-pyridone (AKF-BR) or PIRFENIDONE (PFD) in 10% serum medium were added into each well with 5 replicates for each concentration. The cells were stained with MTT (10 μL per well) at 24, 48, and 72 hours post drug treatment. After 4 hrs of incubation, the medium with MTT was aspirated from each well. One hundred μL MTT solvent was added to each well for 15 min. The dissolved MTT was then measured with a plate reader at 570 nm.

The results are shown in Table 1. Both 1-(3′-fluorophenyl)-5-methyl-2(1H)-pyridone (AKF-PD) and 1-(3′-bromophenyl)-5-methyl-2(1H)-pyridone (AKF-BR) were capable of inhibiting the proliferation of kidney fibroblast. The inhibitory effect was AKF-PD>AKF-BR>pirfenidone. AKF-PD showed a significantly stronger inhibitory effect than pirfenidone.

TABLE 1
Effects of AKF-PD, AKF-BR and PFD on Kidney Fibroblast

| | | Optical Density at 570 nm | | |
|---|---|---|---|---|
| Group | | 24 h | 48 h | 72 h |
| Control | | 0.5978 ± 0.0143 | 0.5994 ± 0.0124 | 0.6338 ± 0.0095 |
| AKF-PD 100 | μg/ml | 0.5907 ± 0.0199 | 0.5850 ± 0.0134 | 0.5080 ± 0.0145* |
| AKF-PD 500 | μg/ml | 0.5799 ± 0.1086 | 0.5185 ± 0.0331 | 0.4314 ± 0.0264*# |
| AKF-PD 1000 | μg/ml | 0.5638 ± 0.0142* | 0.4298 ± 0.0258*# | 0.3511 ± 0.0215*# |
| AKF-BR 100 | μg/ml | 0.5937 ± 0.0307 | 0.5811 ± 0.0161 | 0.5363 ± 0.0158* |
| AKF-BR 500 | μg/ml | 0.5895 ± 0.0253 | 0.5418 ± 0.0221* | 0.4731 ± 0.0249* |
| AKF-BR 1000 | μg/ml | 0.5723 ± 0.0254 | 0.5192 ± 0.0178* | 0.4161 ± 0.0249* |
| PFD 1000 | μg/ml | 0.5911 ± 0.1002 | 0.5844 ± 0.0171 | 0.5264 ± 0.1530 |
| PFD 500 | μg/ml | 0.5877 ± 0.1204 | 0.5450 ± 0.0196* | 0.4798 ± 0.2355* |
| PFD 100 | μg/ml | 0.5798 ± 0.0149 | 0.5272 ± 0.0229* | 0.4269 ± 0.0302* |

*p < 0.05 vs control;
#p < 0.05 vs PFD

### EXAMPLE 2

### Suppression of Rat Myocardiac Fibroblasts Proliferation by 1-(3′-Fluorophenyl)-5-Methyl-2(1H)-Pyridone and PIRFENIDONE

Cell proliferation was measured by MTT assay. DMEM with 10% fetal serum was used as cell culture medium. The cells were prepared in suspension (1×10⁵/ml), and 100 μL of the suspension was transferred to each well of a 96 wells plate. Once the cells were attached to the plastic, the culture was changed to serum free medium and incubated for another 24 hours. Then, the serum free medium was aspirated, and various concentrations of 1-(3′-fluorophenyl)-5-methyl-2(1H)-pyridone (AKF-PD) or PIRFENIDONE (PFD) in 10% serum medium were added into each well with 5 replicates for each concentration. The cells were stained with MTT (10 μL per well) at 12, 24, or 48 hours post drug treatment. After 4 hrs of incubation, the medium with MTT was aspirated from each well. One hundred μL MTT solvent was added to each well for 15 min, and the dissolved MTT was measured with a plate reader at 570 nm.

The results are shown in Table 2. At concentrations of 100 μg/ml, 500 μg/ml, 1000 μg/ml and 2500 μg/ml, both AKF-PD and PIRFENIDONE can suppress proliferation of rat myocardiac fibroblasts after 24 hours treatment; however, at 1000 μg/ml and 2500 μg/ml levels, AKF-PD was more potent than PIRFENIDONE. At the same concentration ranges, both AKF-PD and PIRFENIDONE showed suppressive effects on cell proliferation after 48 hours, but AKF-PD was more potent at 100 μg/ml, 500 μg/ml, and 1000 μg/ml. In conclusion, AKF-PD is a more potent anti-proliferative agent than PIRFENIDONE on rat myocardiac fibroblasts.

TABLE 2
Effects of AKF-PD and PFD on Rat Myocardiac Fibroblasts

| | Optical Density at 570 nm | | |
|---|---|---|---|
| Group | 12 h | 24 h | 48 h |
| control | 0.330 ± 0.002 | 0.445 ± 0.016 | 0.684 ± 0.008 |
| AKF-PD | 0.328 ± 0.010 | 0.426 ± 0.006* | 0.620 ± 0.018*** |
| 100 μg/ml | | | |
| AKF-PD | 0.326 ± 0.003 | 0.408 ± 0.009** | 0.580 ± 0.014*** |
| 500 μg/ml | | | |
| AKF-PD | 0.332 ± 0.006 | 0.392 ± 0.008** | 0.538 ± 0.009*** |
| 1000 μg/ml | | | |
| AKF-PD | 0.325 ± 0.008 | 0.377 ± 0.013*** | 0.514 ± 0.005*** |
| 2500 μg/ml | | | |
| PFD | 0.330 ± 0.014 | 0.429 ± 0.009* | 0.654 ± 0.007*⁺ |
| 100 μg/ml | | | |
| PFD | 0.329 ± 0.013 | 0.411 ± 0.006* | 0.612 ± 0.014***⁺⁺ |
| 500 μg/ml | | | |
| PFD | 0.331 ± 0.009 | 0.403 ± 0.010**⁺ | 0.597 ± 0.013***⁺⁺⁺ |
| 1000 μg/ml | | | |
| PFD | 0.329 ± 0.008 | 0.392 ± 0.009**⁺ | 0.566 ± 0.027** |
| 2500 μg/ml | | | |

*p < 0.05 vs control;
^{+p < 0.05 vs AKF-PD;}
**p < 0.01 vs control;
^{++p < 0.01 vs AKF-PD;}
***p < 0.001 vs control;
^{+++p < 0.001 vs AKF-PD;}

### EXAMPLE 3

### Suppression of Human Stellate Cell Proliferation by 1-(3′-Fluorophenyl)-5-Methyl-2(1H)-Pyridone and PIRFENIDONE

Cell proliferation was measured by MTT assay. DMEM with 10% fetal serum was used as cell culture medium. The cells were prepared in suspension (1×10⁵/ml), and 100 μL of the suspension was transferred to each well of a 96 wells plate. Once the cells were attached to the plastic, the culture was changed to serum free medium and incubated for another 24 hours. Then, the serum free medium was aspirated, and various concentrations of 1-(3′-fluorophenyl)-5-methyl-2(1H)-pyridone (AKF-PD) or PIRFENIDONE (PFD) in 10% serum medium were added into each well with 5 replicates for each concentration. The cells were stained with MTT (10 μL per well) at 12, 24, or 48 hours post drug treatment. After 4 hrs of incubation, the medium with MTT was aspirated from each well. One hundred μL MTT solvent was added to each well for 15 min, and the dissolved MTT was then measured with a plate reader at 570 nm.

The results are shown in Table 3. At 500 μg/ml, 1000 μg/ml, and 2500 μg/ml, both AKF-PD and PIRFENIDONE can suppress proliferation of human stellate cells beginning from 12 hours post drug treatment. At the 24 hours time point, 1000 μg/ml and 2500 μg/ml of AKF-PD was more suppressive than PIRFENIDONE. At 48 hours, AKF-PD was more suppressive than PIRFENIDONE at concentrations of 500 μg/ml, 1000 μg/ml, and 2500 μg/ml. In conclusion, AKF-PD is a more potent anti-proliferative agent than PIRFENIDONE on human stellate cells.

TABLE 3
Effects of AKF-PD and PFD on Human Stellate Cells

| | | Optical Density at 570 nm | | |
|---|---|---|---|---|
| Group | | 12 h | 24 h | 48 h |
| Control | | 0.207 ± 0.001 | 0.370 ± 0.002 | 0.455 ± 0.002 |
| AKF-PD 100 | μg/ml | 0.202 ± 0.001 | 0.366 ± 0.002 | 0.442 ± 0.006 |
| AKF-PD 500 | μg/ml | 0.202 ± 0.001* | 0.341 ± 0.003** | 0.406 ± 0.002*** |
| AKF-PD 1000 | μg/ml | 0.198 ± 0.001** | 0.312 ± 0.003*** | 0.385 ± 0.004*** |
| AKF-PD 2500 | μg/ml | 0.195 ± 0.002** | 0.273 ± 0.005*** | 0.246 ± 0.001*** |
| PFD 100 | μg/ml | 0.206 ± 0.003 | 0.371 ± 0.001 | 0.447 ± 0.003 |
| PFD 500 | μg/ml | 0.202 ± 0.001* | 0.345 ± 0.002** | 0.413 ± 0.001***⁺⁺ |
| PFD 1000 | μg/ml | 0.201 ± 0.001* | 0.330 ± 0.001***⁺⁺ | 0.402 ± 0.001***⁺⁺ |
| PFD 2500 | μg/ml | 0.198 ± 0.001** | 0.278 ± 0.001***⁺ | 0.306 ± 0.002***⁺⁺⁺ |

*p < 0.05 vs control;
^{+p < 0.05 vs AKF-PD;}
**p < 0.01 vs control;
^{++p < 0.01 vs AKF-PD;}
***p < 0.001 vs control;
^{+++p < 0.001 vs AKF-PD;}

### EXAMPLE 4

### Suppression of Rat Pulmonary Fibroblast Proliferation by 1-(3′-Fluorophenyl)-5-Methyl-2(1H)-Pyridone, 1-(3′-bromophenyl)-5-Methyl-2(1H)-Pyridone and PIRFENIDONE

Cell proliferation was measured by MTT assay. DMEM with 10% fetal serum was used as cell culture medium. The cells were prepared in suspension (1×10⁵/ml), and 100 μL of the suspension was transferred to each well of a 96 wells plate. Once the cells were attached to the plastic, the culture was changed to serum free medium and incubated for another 24 hours. Then, the serum free medium was aspirated, and various concentrations of 1-(3′-fluorophenyl)-5-methyl-2(1H)-pyridone (AKF-PD) or 1-(3′-bromophenyl)-5-Methyl-2(1H)-Pyridone or PIRFENIDONE (PFD) in 10% serum medium were added into each well with 5 replicates for each concentration. The cells were stained with MTT (10 μL per well) at 24, or 48 hours post drug treatment. After 4 hrs of incubation, the medium with MTT was aspirated from each well. One hundred μL MTT solvent was added to each well for 15 min, and the dissolved MTT was measured with a plate reader at 570 nm.

The results are shown in Table 4. Both 1-(3′-fluorophenyl)-5-methyl-2(1H)-pyridone (AKF-PD) and 1-(3′-bromophenyl)-5-methyl-2(1H)-pyridone (AKF-BR) could suppress proliferation of rat pulmonary fibroblasts. The inhibitory effect among the test compounds was AKF-PD>AKF-BR>pirfenidone. AKF-PD has a significant stronger inhibitory effect than pirfenidone does.

TABLE 4
Effects of AKF-PD, AKF-BR and PFD on Rat Pulmonary Fibroblasts

Optical Density at 570 nm

| Group | | 24 h | 48 h |
|---|---|---|---|
| Control | | 0.1713 ± 0.0226 | 0.1754 ± 0.0167 |
| AKF-PD 100 | μg/ml | 0.1467 ± 0.0138* | 0.1369 ± 0.0115*^{#} |
| AKF-PD 500 | μg/ml | 0.1258 ± 0.0119* | 0.1214 ± 0.0234*^{#} |
| AKF-PD 1000 | μg/ml | 0.1130 ± 0.0163* | 0.1119 ± 0.0285*^{#} |
| AKF-BR 100 | μg/ml | 0.1654 ± 0.0143 | 0.1475 ± 0.0211* |
| AKF-BR 500 | μg/ml | 0.1342 ± 0.0237* | 0.1292 ± 0.0178*^{#} |
| AKF-BR 1000 | μg/m1 | 0.1204 ± 0.0176* | 0.1201 ± 0.0342*^{#} |
| PFD 100 | μg/ml | 0.2023 ± 0.0169 | 0.1864 ± 0.0530 |
| PFD 500 | μg/ml | 0.1887 ± 0.0130 | 0.1459 ± 0.0255* |
| PFD 1000 | μg/m1 | 0.1797 ± 0.0166 | 0.1269 ± 0.0302* |

*p < 0.05 vs control;
^{#p < 0.05 vs PFD}

### EXAMPLE 5

### Suppression of Human Skin Scar Forming Fibroblast Proliferation by 1-(3′-Fluorophenyl)-5-Methyl-2(1H)-Pyridone and PIRFENIDONE

Cell proliferation was measured by MTT assay. DMEM with 10% fetal serum was used as cell culture medium. The cells were prepared in suspension (1×10⁵/ml), and 100 μL of the suspension was transferred to each well of a 96 wells plate. Once the cells were attached to the plastic, the culture was changed to serum free medium and incubated for another 24 hours. Then, the serum free medium was aspirated, and various concentrations of 1-(3′-fluorophenyl)-5-methyl-2(1H)-pyridone (AKF-PD) or PIRFENIDONE (PFD) in 10% serum medium were added into each well with 5 replicates for each concentration. The cells were stained with MTT (10 μL per well) at 12, 24, or 48 hours post drug treatment. After 4 hrs of incubation, the medium with MTT was aspirated from each well. One hundred μL MTT solvent was added to each well for 15 min, and the dissolved MTT was measured with a plate reader at 570 nm.

The results are shown in Table 5. After drug treatment for 24 hrs at concentrations of 100 μg/ml, 500 μg/ml, 1000 μg/ml, and 2500 μg/ml, both AKF-PD and PIRFENIDONE were capable of inhibiting the growth of human skin fibroblasts; however, AKF-PD was more potent than PIRFENIDONE at concentrations of 500 μg/ml, 1000 μg/ml, and 2500 μg/ml. After 48 hrs of treatment, 500 μg/ml or 1000 μg/ml of AKF-PD showed more inhibition than similar concentrations of PIRFEIDONE. In conclusion, AKF-PD is a more potent anti-proliferative agent than PIRFENIDONE on human skin fibroblasts.

TABLE 5
Effects of AKF-PD and PFD on Human Skin Scar Forming Fibroblasts

| | Optical Density at 570 nm | | |
|---|---|---|---|
| Group | 12 h | 24 h | 48 h |
| Control | 0.195 ± 0.008 | 0.263 ± 0.005 | 0.381 ± 0.001 |
| AKF-PD | 0.192 ± 0.010 | 0.245 ± 0.002* | 0.366 ± 0.006* |
| 100 μg/ml | | | |
| AKF-PD | 0.192 ± 0.006 | 0.238 ± 0.004* | 0.345 ± 0.007* |
| 500 μg/ml | | | |
| AKF-PD | 0.192 ± 0.009 | 0.221 ± 0.004** | 0.323 ± 0.009*** |
| 1000 μg/ml | | | |
| AKF-PD | 0.190 ± 0.002 | 0.198 ± 0.008*** | 0.267 ± 0.001*** |
| 2500 μg/ml | | | |
| PFD | 0.194 ± 0.004 | 0.250 ± 0.003* | 0.366 ± 0.006* |
| 100 μg/ml | | | |
| PFD | 0.191 ± 0.008 | 0.245 ± 0.004*⁺ | 0.350 ± 0.003***⁺⁺ |
| 500 μg/ml | | | |
| PFD | 0.190 ± 0.008 | 0.330 ± 0.001*⁺⁺ | 0.328 ± 0.004***⁺⁺ |
| 1000 μg/ml | | | |
| PFD | 0.193 ± 0.004 | 0.278 ± 0.001***⁺⁺⁺ | 0.264 ± 0.005*** |
| 2500 μg/ml | | | |

*p < 0.05 vs control;
^{+p < 0.05 vs AKF-PD;}
**p < 0.01 vs control;
^{++p < 0.01 vs AKF-PD;}
***p < 0.001 vs control;
^{+++p < 0.001 vs AKF-PD;}

### EXAMPLE 6

### Suppression of Human Peritoneal Mesothelial Cell Proliferation by 1-(3′-Fluorophenyl)-5-Methyl-2(1H)-Pyridone and PIRFENIDONE

Cell proliferation was measured by MTT assay. DMEM with 10% fetal serum was used as cell culture medium. The cells were prepared in suspension (1×10⁵/ml), and 100 μL of the suspension was transferred to each well of a 96 wells plate. Once the cells were attached to the plastic, the culture was changed to serum free medium and incubated for another 24 hours. Then, the serum free medium was aspirated, and various concentrations of 1-(3′-fluorophenyl)-5-methyl-2(1H)-pyridone (AKF-PD) or PIRFENIDONE (PFD) in 10% serum medium were added into each well with 5 replicates for each concentration. The cells were stained with MTT (10 μL per well) at 12, 22, or 48 hours post drug treatment. After 4 hrs of incubation, the medium with MTT was aspirated from each well. One hundred μL MTT solvent was added to each well for 15 min, and the dissolved MTT was measured with a plate reader at 570 nm.

The results are shown in Table 6. After drug treatment for 24 hrs at concentrations of 500 μg/ml, 1000 μg/ml, and 2500 μg/ml, both AKF-PD and pirfenidone were capable of inhibiting the growth of human peritoneal mesothelial cells; however, AKF-PD was more potent than PIRFENIDONE at concentrations of 1000 μg/ml and 2500 μg/ml. For 48 hrs of treatment, 1000 μg/ml of AKF-PD showed more inhibition than similar concentration of PIRFEIDONE. In conclusion, AKF-PD is a more potent anti-proliferative agent than PIRFENIDONE on human peritoneal mesothelial cells.

TABLE 6
Effects of AKF-PD and PFD on Human Peritoneal Mesothelial Cells

| | Optical Density at 570 nm | | |
|---|---|---|---|
| Group | 12 h | 24 h | 48 h |
| Control | 0.347 ± 0.006 | 0.585 ± 0.002 | 0.814 ± 0.003 |
| AKF-PD | 0.344 ± 0.004 | 0.583 ± 0.004 | 0.807 ± 0.007 |
| 100 μg/ml | | | |
| AKF-PD | 0.344 ± 0.005 | 0.573 ± 0.004* | 0.758 ± 0.010* |
| 500 μg/ml | | | |
| AKF-PD | 0.343 ± 0.004 | 0.553 ± 0.004*** | 0.704 ± 0.003*** |
| 1000 μg/ml | | | |
| AKF-PD | 0.346 ± 0.005 | 0.502 ± 0.003*** | 0.646 ± 0.006*** |
| 2500 μg/ml | | | |
| PFD | 0.346 ± 0.006 | 0.584 ± 0.005 | 0.810 ± 0.006 |
| 100 μg/ml | | | |
| PFD | 0.343 ± 0.004 | 0.577 ± 0.003* | 0.766 ± 0.004*** |
| 500 μg/ml | | | |
| PFD | 0.363 ± 0.003 | 0.563 ± 0.003***⁺ | 0.714 ± 0.002***⁺⁺⁺ |
| 1000 pg/ml | | | |
| PFD | 0.345 ± 0.005 | 0.512 ± 0.006***⁺ | 0.648 ± 0.009*** |
| 2500 pg/ml | | | |

*p < 0.05 vs control;
^{+p < 0.05 vs AKF-PD;}
**p < 0.01 vs control;
^{++p < 0.01 vs AKF-PD;}
***p < 0.001 vs control;
^{+++p < 0.001 vs AKF-PD;}

### EXAMPLE 7

### Effects of 1-(3′-Fluorophenyl)-5-Methyl-2(1H)-Pyridone (AKF-PD) on Glomerulosclerosis and Interstitial Renal Fibrosis

Rat diabetic kidney disease was induced by streptozotocin (STZ) to test the effectiveness of 5-methyl-2(1H)-pyridone (AKF-PD) in preventing glomerulosclerosis and renal interstitial fibrosis.

Eight-week old male Wistar rat (180 g˜220 g) were randomly grouped as normal, diabetic nephrosis, diabetic nephrosis/valsartan, or diabetic nephrosis/AKF-PD. Diabetes was induced by a single i.p. injection of STZ 55 mg/kg. Diabetic condition was confirmed 24 hours later by the following test results: 13.9 mmol/L of fasting blood-glucose, 16.7 mmol/L random blood-glucose, and positive urine-glucose. If the diabetic rat had urine protein level higher than 30 mg/d 4 weeks later, a rat model was established for diabetic nephrosis.

Rats in the group of diabetic nephrosis/AKF-PD were orally fed 500 mg/kg/d of AKF-PD, and rats in the group of diabetic nephrosis/valsartan were fed 30 mg/kg/d of valsartan. Normal saline was fed to rats in the group of diabetic nephrosis. After 12 weeks of drug treatment, all rats were sacrificed and their kidneys were removed for pathological examination. The references for standard histopathological scoring system for glomerulus and renal tubules interstitial tissue are: Radford et al., Predicting renal outcome in IgA nephropathy. J. Am. Soc. Nephrol. 8(2) 199-207(1997); Zhao et al., Comparison of renoprotective effect between Angiotensin II receptor antagonist and angiotensin-converting enzyme inhibitor on puromycin nephropathy and their possible mechanism. Chin. J. Mult. Organ Dis. Elderly 1(1) 36-40(2002).

The results of microscopic examination of kidney tissues are shown in Table 7. Compared to diabetic nephrosis rats without any treatment, the AKF-PD-treated rats showed less damage on their glomerulus and renal tubules interstitial tissue, indicating that AKF-PD may effectively treat glomerulus sclerosis and renal tubules interstitial fibrosis caused by diabetic condition.

TABLE 7
Histopathological Scores From Different Treatment Groups

| | | | Renal Tubules |
|---|---|---|---|
| | Number of | | Interstitial Tissue |
| Groups | Animals | Glomerular Score | Score |
| Diabetic | 5 | 1.29 +− 0.18 | 2.20 +− 0.14 |
| Nephrosis + | | | |
| AKF-PD | | | |
| Diabetic | 8 | 1.36 +− 0.24 | 3.12 +− 0.64 |
| Nephrosis + | | | |
| Valsartan | | | |
| Diabetic | 9 | 1.63 +− 0.33 | 3.33 +− 0.54 |
| Nephrosis | | | |
| Normal Rat | 5 | 0.21 +− 0.04 | 0.48 +− 0.14 |

### EXAMPLE 8

### Effects of 1-(3′-Fluorophenyl)-5-Methyl-2(1H)-Pyridone (AKF-PD) in a Rat Model of Renal Interstitial Fibrosis

Anti-fibrotic effect of AKF-PD was tested on a SD rat model for renal interstitial fibrosis induced by surgical ligation of single side ureter (Unilateral Ureteral Obstruction, UUO, model). Eight weeks old male SD rats (180 g˜220 g) were randomly divided into sham surgical group, disease model group, Enalapril (10 mg/kg/d) group and AKF-PD (500 mg/kg/d) group. Under aseptic condition, all animals in the groups of disease model, Enalapril and AKF-PD had a surgical procedure for ligation of the left side ureter. The animals of the sham surgical group experienced the same surgical procedure except the ligation step. The respective drugs were administrated by gavage to rats in the groups of Enalapril and AKF-PD from one day prior to the procedure to 14 days post surgical procedure. Normal saline was administrated in a similar fashion to the rats in the groups of disease model and sham surgical. The animals were sacrificed 14 days after surgical procedure and their left kidney were removed for pathological (HE staining) examination. Histological scoring for interstitial compartment was done according to Radford's method (Radford et al., Predicting renal outcome in IgA nephropathy. J. Am. Soc. Nephrol. 8: 199-207(1997)). As shown in Table 8, rats treated with AKF-PD showed reduced lesion on interstitial tissues comparing to those in the groups of disease model and Enalapril, indicating AKF-PD may be an effective drug for interstitial renal fibrosis.

TABLE 8
Comparison of Histological Scores for Interstitial Compartment

| | Sham | Disease | | |
|---|---|---|---|---|
| Group | surgical | Model | Enalapril | AKF-PD |
| Number of rats | 10 | 9 | 9 | 8 |
| (n) | | | | |
| Score | 1.33 ± 0.58 | 10.24 ± 0.99 | 9.21 ± 0.64 | 6.43 ± 1.28 |

### EXAMPLE 9

### Inhibition of TGF-β Production And Type I & III Collagen Accumulation In Rat UUO Model (Unilateral Ureteral Obstruction) by 1-(3′-Fluorophenyl)-5-Methyl-2(1H)-Pyridone (AKF-PD)

Similar to the example 8, Kidney fibrosis was induced by Unilateral Ureteral Obstruction. And the animal experiments were performed to investigate whether AKF-PD has any effect on TGF-β production and collagen accumulation. These are two known factors for fibrotic lesion.

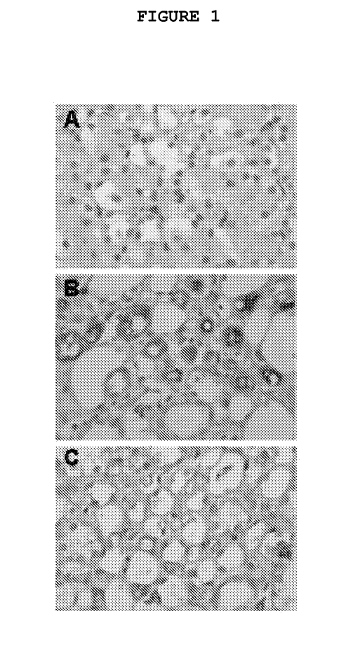

As shown by FIG. 1, sections A-C were immunohistochemically stained with specific antibody against TGF-β with brown color indicating TGF-β protein expression in kidney cells. All sections were also counterstained lightly with hematoxylin for viewing non-TGF-β stained cells. The result indicates that the administration of AKF-PD to the fibrotic rats have significantly reduced TGF-β production.

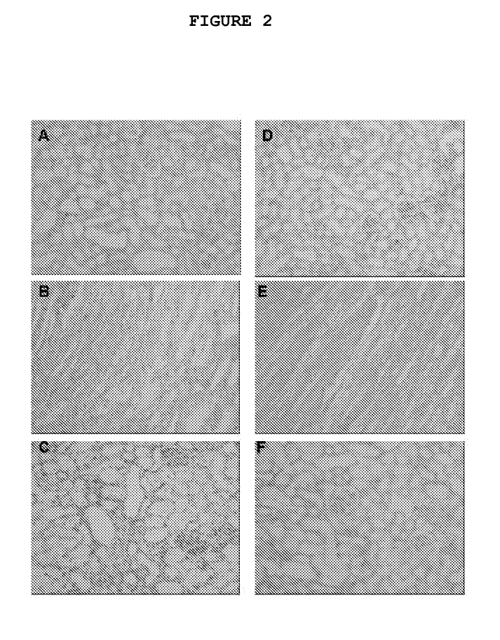

Also shown by FIG. 2, type I and III collagen were analyzed by immunohistochemistry. The typical fibrotic fiber was viewed as brown color. There are a robotic accumulation of both type I (B) and type III (E) collagen on the fibrotic kidney of UUO model. However, treatment of the diseased rat with AKF-PD significantly blocks type I and III collagen accumulation or production. Reduced collagen level can be interpreted as the reduced ECM.

The decreased TGF-β production and reduced accumulation of ECM indicates a possible therapeutic effect of AKF-PD for kidney fibrotic condition.

### EXAMPLE 10

### Effects of 1-(3′-Fluorophenyl)-5-Methyl-2(1H)-Pyridone (AKF-PD) on Liver Fibrosis

Kun Ming (KM) mice were infected with Schistosoma miracidium to induce schistosomial liver fibrosis. Four to six weeks old male Kun Ming (KM) mice (18-22 g) were randomly grouped into healthy, infected, infected/Pyquiton, infected/γ-interferon, and infected/AKF-PD groups. Ten Schistosoma miracidium were placed on shaved abdominal skin of each mouse for infection. Eight weeks post-infection, mice in the groups of infected/Pyquiton, infected/γ-interferon, and infected/AKF-PD were disinfected by treating with 650 mg/kg Pyquiton for 4 days. Five hundred mg/kg AKF-PD were given daily by gavage to the disinfected mice of infected/AKF-PD group. The disinfected mice were given (i.m.) 50,000 unit γ-interferon daily. The disinfected mice in the infected/Pyquiton and infected groups were orally given normal saline once a day in the same way as administration of drug treatment. Drug or normal saline treatment was continued for 8 weeks. All mice were sacrificed one week after discontinue of drug treatment, and the left lobe of liver from each mouse was taken for pathological examination.

Examination of HE-stained slices of liver tissue were carried out as follows. In general, after 16 weeks of Schistosoma miracidia infection, the area of schistosomo egg granuloma would directly correlate to the severity of liver fibrosis. Therefore, the area of schistosomo egg granuloma was measured using a high-resolution, color pathology graphic analyzer (HPIAS-1000). The sum of area of 5 granuloma with abundant eggs and the sum of area of 5 granuloma with few eggs were measured for each slice. As shown in Tables 9 and 10, AKF-PD-treated animals had smaller area (μm²) of schistosomo egg granuloma than those in either no-treatment (infected only) or Pyquiton-treated group.

TABLE 9
Comparison of Schistosomo Egg Granuloma Area

| | | Number | | |
|---|---|---|---|---|
| | Group of | of | Mean | |
| Granuloma | animal/treatment | animal | (μm²) | S.D. |
| With | Infected/γ-interferon | 9 | 83706.79 | 22943.48 |
| Abundant | Infected/AKF-PD. | 10 | 80155.11 | 25419.82 |
| Eggs | Infected only | 10 | 111604.59 | 30115.49 |
| | Infected/Pyquiton | 11 | 125823.35 | 31708.85 |
| With | Infected/γ-interferon | 9 | 32407.14 | 10078.30 |
| Few | Infected/AKF-PD. | 10 | 30266.68 | 11069.89 |
| Eggs | Infected only | 10 | 41116.13 | 11246.94 |
| | Infected/Pyquiton | 11 | 45418.59 | 18001.53 |

TABLE 10
P values for Groups in Table 9

| | With abundant | With few |
|---|---|---|
| Groups | eggs | eggs |
| Infected only vs infected/γ- | 0.038 | 0.095 |
| interferon, | | |
| infected/γ-interferon, vs | 0.004 | 0.058 |
| infected/Pyquiton | | |
| infected/Pyquiton vs infected/AKF- | 0.002 | 0.032 |
| PD. | | |
| Infected only vs infected/Pyquiton | 0.306 | 0.516 |
| Infected only vs infected/AKF-PD. | 0.021 | 0.043 |
| infected/γ-interferon vs | 0.754 | 0.666 |
| infected/AKF-PD | | |

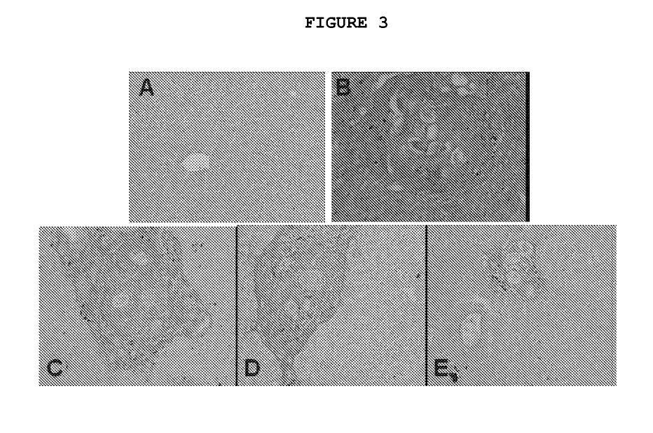

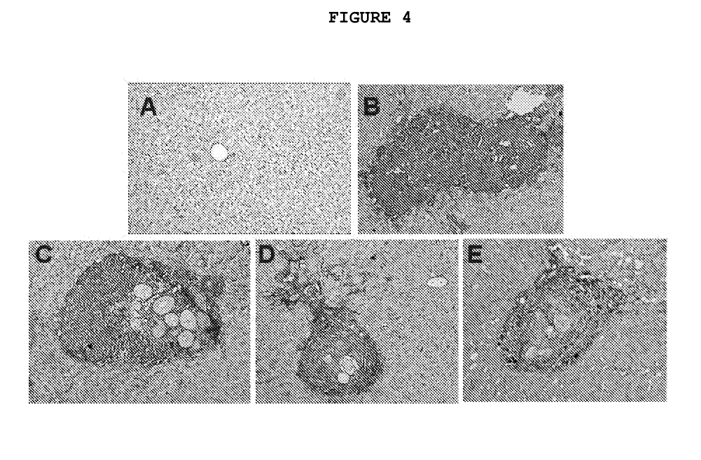

Some representative immunohistological staining for fibrotic nodule and type I collagen were presented in FIG. 3 and 4. As the indicated by FIG. 3, AKF-PD treated rat has much reduced fibrotic nodule than pyquiton and Interferon-γ treated rat. Also as shown by FIG. 4, AKF-PD treated rat has much less type I collagen accumulation than by pyquiton and Interferon-γ treated rat. These results indicate that AKF-PD may be an effective pharmacological agent to treat schistosoma liver fibrosis.

### EXAMPLE 11

### Effects of 1-(3′-Fluorophenyl)-5-Methyl-2(1H)-Pyridone (AKF-PD) on Pulmonary Fibrosis

Bleomycin-induced rat pulmonary fibrosis is selected for testing 5-methyl-1-(3′-fluorophenyl)-2(1H)-pyridone (AKF-PD). Male Sprague-Dawley rats (6-8 weeks old, 180 g˜220 g) were cared under regular condition. The animals were randomly divided into 3 groups: sham surgical group, model disease group, and disease/AKF-PD group. Six mg/kg/4 ml of Bleomycin was slowly infused into the trachea of rats in the model disease and disease/AKF-PD groups. Same amount of normal saline was infused into the trachea of rats in the sham surgical group.

Five hundred mg/kg AKF-PD was directly administrated by gavage to rats in the disease/AKF-PD group daily from 2 days prior to operation through 27 days after operation. Normal saline was used for all animals in both disease and sham surgical groups. The animals were sacrificed at 27 days post surgery and the lung tissues were removed for pathological sample preparation. The HE-stained lung tissues from each rat were examined under microscope to determine the frequency and severity of fibrotic lesion. Evaluation of lung tissue fibrosis was carried out according to the method of Szapiel et al., Bleomycin-induced interstitial pulmonary disease in the nude, athymic mouse. Am. Rev. Respir. Dis. 120:893-9(1979). As shown in Table 11, rats treated with AKF-PD showed less severe fibrotic lesions as compared to those in the disease group, indicating AKF-PD may be an effective agent for treating pulmonary fibrosis disease.

TABLE 11
Comparison of Frequency and Severity of Fibrotic Lesions

| Severity of | AKF-PD group | Disease group | Sham surgical |
|---|---|---|---|
| Fibrosis | Total of 8 rats | Total of 6 rats | Total of 7 rats |
| None | 0 | 0 | 2 |
| Milder | 6 | 1 | 5 |
| Moderate | 1 | 4 | 0 |
| Sever | 1 | 1 | 0 |

### EXAMPLE 12

### Efficacy Comparison of 1-(3′-Fluorophenyl)-5-methyl-2(1H)-pyridone (AKF-PD) and PIRFENIDONE In Kidney Fibrosis And Pulmonary Fibrosis Models

Kidney Fibrosis

Anti-fibrotic effect of AKF-PD was tested on a SD rat model for renal interstitial fibrosis induced by surgical ligation of single side ureter. Male SD rats (180 g˜220 g) were randomly divided into disease model group, PFD (500 mg/kg/d) group and AKF-PD (500 mg/kg/d) group. Under aseptic condition, all animals in the groups of disease model, PFD and AKF-PD had a surgical procedure for ligation of ureter. The respective drugs were administrated by gavage to rats in the groups of PFD and AKF-PD from one day prior to the procedure to 14 days post surgical procedure. Normal saline was administrated in a similar fashion to the rats in the groups of disease model. The animals were sacrificed 14 days after surgical procedure and their kidney were removed for pathological (HE staining) examination. Histological scoring for interstitial compartment was done according to Radford's method (Radford et al., Predicting renal outcome in IgA nephropathy. J. Am. Soc. Nephrol. 8 : 199-207 (1997). As shown in Table 12, rats treated with AKF-PD showed more reduced lesion on interstitial tissues comparing to those in the groups of PFD treated. Although not significantly, the data indicates that AKF-PD may be more effective than PFD for interstitial renal fibrosis.

TABLE 12
Comparison of Histological Scores for Interstitial Compartment

| | Disease | | |
|---|---|---|---|
| Group | Model | PFD | AKF-PD |
| Number of rats | 8 | 8 | 9 |
| (n) | | | |
| Score | 9.53 ± 1.75 | 8.06 ± 2.41 | 7.11 ± 1.38 |

Pulmonary Fibrosis

Under regular care condition, 7-10 weeks old, male, institute of cancer research (ICR) mice, weight 28-39 g, were randomly grouped as normal control, disease, disease/AKF-PD, disease/PFD and disease Captopril group.

Control group was injected with normal saline once day from day 2 to 15 days. Disease, disease/AKF-PD, disease/PFD and disease/captopril groups were injected with bleomycin once day from day 2 to 15. Control and disease groups were given 0.5% CMC by gavage once a day from day 1 to day 28. 500 mg/kg of AKF-PD CMC suspension and PD CMC suspension and 12.5 mg/kg captopril in saline were administered to respective groups by gavage once day from day 1 to day 28. All animals were sacrificed on day 29 and their lungs were removed for pathological (HE staining) examination.

Histological scoring for the lung tissue was done according Szapiel's method. As shown in Table 13, both groups treated with AKF-PD and PFD showed reduced lesion comparing to those in the group of disease model and captopril group (a regular treatment for fibrosis). The results indicate that AKF-PD is similar to PFD in slowing pulmonary fibrosis.

TABLE 13
Histological Score for Lung Tissue

| | | | Std. |
|---|---|---|---|
| Group | N | Mean | Deviation |
| Disease | 6 | 3.0000 | .00000 |
| Disease/Captopril | 7 | 2.7143 | 0.48795 |
| Disease/AKF-PD | 11 | 2.0000 | .77460 |
| Disease/PFD | 11 | 2.0909 | .70065 |

T test

| | |
|---|---|
| disease/AKF-PD VS disease | P = 0.003 |
| disease/AKF-PD VS disease/PDF | P = 0.736 (insignificant) |
| disease/PFD VS disease | P = 0.006 |
| Disease/Captopril VS disease | P = 0.418 (insignificant) |

### EXAMPLE 13

### Comparison of Acute Toxicity of 1-(3′-Fluorophenyl)-5-methyl-2(1H)-pyridone (AKF-PD) and PIRFENIDONE

Male and female Kun Ming (KM) mice weighing between 18 g-22 g were acquired from the animal facility of Hsiang-Ya Medical College, the Central South University. Fifty Kun Ming mice were randomly assigned into 5 groups with 5 male and 5 female mice for each group. The animals went through fasting with a normal water supply before starting drug treatment (either AKF-PD or PIRFENIDONE). The drug was administrated orally by gavage. The volume of liquid drug was 20 ml/kg body weight. The range of dosage for AKF-PD administrated was from 1071 mg/kg to 6000 mg/kg. The dosage difference between two adjacent doses was 1:0.65 (a dose vs the next lower dose). All animals were maintained under a regular condition. Acute toxic reaction and death within 14 days of post drug treatment were recorded. Autopsy was performed on all dead animals and visual examination was performed on all organs.

LD₅₀ was calculated according to Bliss method. Acute toxicity LD₅₀ for 1-(3′-fluorophenyl)-5-methyl-2(1H)-pyridone was 2979.89 mg/kg with a 95% confidence limit of 2402.70-3695.73 mg/kg (Table 14). LD₅₀ for PIRFENIDONE was 955.4 mg/kg with a 95% confidence limit of 550.9-1656.7 mg/kg (Table 15). The results of Table 15 are very close to those reported in the literature: 997.7 mg/kg (U.S. Pat. No. 5,310,562) and 1112 mg/kg(Pharmaceutical Care and Research 5:4823(2005)). These results indicate that the toxicity for AKF-PD is only one third of that of PIRFENIDONE (2978 vs 955).

TABLE 14
LD₅₀ of 1-(3′-Fluorophenyl)-5-methyl-2(1H)-pyridone

| | Dosage in | | Number | Percentage | |
|---|---|---|---|---|---|
| Dosage | log scale | Number | of | of death | LD₅₀ and 95% |
| (mg/kg) | (x) | of Mice | death | (%) | confidence limit |
| 6000.0 | 3.7782 | 10 | 9 | 90 | 2979.89 mg/kg |
| 3900.0 | 3.5911 | 10 | 7 | 70 | (2402.70 − 3695.73) |
| 2535.0 | 3.4040 | 10 | 4 | 40 | |
| 1647.8 | 3.2169 | 10 | 1 | 10 | |
| 1071.0 | 3.0298 | 10 | 0 | 0 | |

TABLE 15
LD₅₀ of PIRFENIDONE

| | Dosage in | Number | Number | Percentage | LD₅₀ and 95% |
|---|---|---|---|---|---|
| Dosage | log scale | of | of | of death | confidence |
| (mg/kg) | (x) | Mice | death | (%) | limit |
| 6000 | 3.778 | 10 | 10 | 100 | 955.4 mg/kg |
| 3900 | 3.591 | 10 | 10 | 100 | (550.9 − 1656.7) |
| 2535 | 3.030 | 10 | 8 | 80 | |
| 1647.8 | 3.404 | 10 | 6 | 60 | |
| 1071 | 3.217 | 10 | 5 | 50 | |

### EXAMPLE 14

### AKF-PD: A Novel Anti-Scarring Therapy for Advanced Diabetic Nephropathy

The study described below will determine whether the experimental drug AKF-PD can slow kidney disease in patients with diabetes. Diabetes can cause accumulation of proteins in the kidneys, leading to scar formation and eventual kidney failure. AKF-PD has been shown to reduce fibrosis in multiple experimental models, including pulmonary fibrosis, liver sclerosis, and renal disease. In animal models of renal diseases, AKF-PD reduces glomerulosclerosis and interstitial fibrosis. It is anticipated that AKF-PD may be able to slow scar formation in diabetic kidney disease and prolong kidney function.

We will enroll 30 adult patients with type 1 or 2 diabetes with glomerular filtration rate (GFR) between 20-75 ml/min/1.73 m², greater than 300 mg/d of proteinuria, and blood pressure less than or equal to 140/90 on an ACE inhibitor or an ARB. Participants are randomly assigned to take either 1200 mg of AKF-PD, 2400 mg of AKF-PD, or a placebo by mouth three times a day for 1 year. They return to the clinic 2 weeks after the initial screening visit and then every 3 months throughout the study for fasting blood and urine tests, blood pressure measurement and reviews of any health-related issues. Additional blood samples may be drawn to see if AKF-PD is affecting the level of certain proteins or other related molecules that are thought to be related to kidney disease progression in diabetes.

Patients are asked to check their blood pressure at home at least 3 times a week and record it in a log. A patient whose blood pressure is greater than 130/80 must call the doctor to adjust his or her medications. Patients may also need to monitor their blood sugar more frequently than usual (up to 4 times a day) and possibly give more frequent insulin injections to achieve good control of their diabetes.

Patients are asked to collect 24-hour urine five times during the study: at baseline, 2 weeks, 6 months, 12 months, and 54 weeks (end of study). In addition, they are seen by an eye doctor at baseline and at the end of the study to evaluate if AKF-PD may be beneficial for eye problems related to diabetes.

Patients will be maintained on the current standard of care for diabetic nephropathy, including an angiotensin converting enzyme (ACE) inhibitor and/or angiotensin receptor blocker (ARB), antihypertensive therapy with blood pressure target of less than 130/80, and aggressive glycemic control with target hemoglobin A1C of less than 7%.

The primary endpoint will be the change in renal function from baseline to the end of the study period. Renal function will be assessed by the GFR. The secondary endpoints will include the percent change in urine albumin excretion and the levels of urine and plasma TGF-β from baseline to the end of the study period.

Based on data from experimental animal models, it is anticipated that AKF-PD will significantly improve renal function and reduce TGF-β levels.

What is claimed is:

1. A composition comprising one or more 5-methyl-1-(substituted phenyl)-2(1H)-pyridone in an amount effective for treating organ or tissue fibrosis, said 5-methyl-1-(substituted phenyl)-2(1H)-pyridone having a formula of: 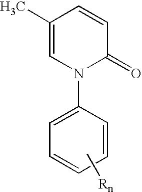
wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

2. The composition of claim 1, wherein the 5-methyl-1-(substituted phenyl)-2(1H)-pyridone is selected from the group consisting of 1-(2′-bromophenyl)-5-methyl-2(1H)-pyridone, 1-(3′-bromophenyl)-5-methyl-2(1H)-pyridone, 1-(4′-bromophenyl)-5-methyl-2(1H)-pyridone, 1-(2′- chlorophenyl)-5-methyl-2(1H)-pyridone, 1-(3′-chlorophenyl)-5-methyl-2(1H)-pyridone, 1-(2′-fluorophenyl)-5-methyl-2(1H)-pyridone, 1-(3′-fluorophenyl)-5-methyl-2(1H)-pyridone, 1-(4′-fluorophenyl)-5-methyl-2(1H)-pyridone, 1-(2′-iodophenyl)-5-methyl-2(1H)-pyridone, 1-(3′-iodophenyl)-5-methyl-2(1H)-pyridone, 1-(4′-iodophenyl)-5-methyl-2(1H)-pyridone, 1-(2′,3′-dibromophenyl)-5-methyl-2(1H)-pyridone, 1-(2′,4′-dibromophenyl)-5-methyl-2(1H)-pyridone, 1-(2′,5′-dibromophenyl)-5-methyl-2(1H)-pyridone, 1-(2′,6′-dibromophenyl)-5-methyl-2(1H)-pyridone, 1-(3′,4′-dibromophenyl)-5-methyl-2(1H)-pyridone, 1-(3′,5′-dibromophenyl)-5-methyl-2(1H)-pyridone, 1-(2′,3′-dichlorophenyl)-5-methyl-2(1H)-pyridone, 1-(2′,4′-dichlorophenyl)-5-methyl-2(1H)-pyridone, 1-(2′,5′-dichlorophenyl)-5-methyl-2(1H)-pyridone, 1-(2′,6′-dichlorophenyl)-5-methyl-2(1H)-pyridone, 1-(3′,4′-dichlorophenyl)-5-methyl-2(1H)-pyridone, 1-(3′,5′-dichlorophenyl)-5-methyl-2(1H)-pyridone, 1-(2′,3′-difluorophenyl)-5-methyl-2(1H)-pyridone, 1-(2′,4′-difluorophenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluorophenyl)-5-methyl-2(1H)-pyridone, 1-(3′,5′-difluorophenyl)-5-methyl-2(1H)-pyridone, 5-methyl-1-(2′-trifluoromethylphenyl)-2(1H)-pyridone, 5-methyl-1-(4′-trifluoromethylphenyl)-2(1H)-pyridone, 1-(2′,3′-bis-trifluoromethylphenyl)-5-methyl-2(1H)-pyridone, 1-(2′,4′-bis-trifluoromethylphenyl)-5-methyl-2(1H)-pyridone, 1-(2′,5′-bis-trifluoromethylphenyl)-5-methyl-2(1H)-pyridone, 1-(2′,6′-bis-trifluoromethylphenyl)-5-methyl-2(1H)-pyridone, 1-(3′,4′-bis-trifluoromethylphenyl)-5-methyl-2(1H)-pyridone, 1-(3′,5′-bis-trifluoromethylphenyl)-5-methyl-2(1H)-pyridone, 5-methyl-1-(2′-methylphenyl)-2(1H)-pyridone, 1-(3′-methylphenyl)-5-methyl-2(1H)-pyridone, 1-(2′,3′-dimethylphenyl)-5-methyl-2(1H)-pyridone, 1-(2′,4′-dimethylphenyl)-5-methyl-2(1H)-pyridone, 1-(2′,5′-dimethylphenyl)-5-methyl-2(1H)-pyridone, 1-(2′,6′-dimethylphenyl)-5-methyl-2(1H)-pyridone, 1-(3′,4′-dimethylphenyl)-5-methyl-2(1H)-pyridone, 1-(3′,5′-dimethylphenyl)-5-methyl-2(1H)-pyridone, 1-(2′-methoxyphenyl)-5-methyl-2(1H)-pyridone, 1-(3′-methoxyphenyl)-5-methyl-2(1H)-pyridone. 1-(2′,3′-dimethoxyphenyl)-5-methyl-2(1H)-pyridone, 1-(2′,4′-dimethoxyphenyl)-5-methyl-2(1H)-pyridone, 1-(2′,5′-dimethoxyphenyl)-5-methyl-2(1H)-pyridone, 1-(2′,6′-dimethoxyphenyl)-5-methyl-2(1H)-pyridone, 1-(3′,4′-dimethoxyphenyl)-5-methyl-2(1H)-pyridone, and 1-(3′,5′-dimethoxyphenyl)-5-methyl-2(1H)-pyridone.

3. The composition of claim 1, wherein the amount effective for treating organ or tissue fibrosis comprises a daily dosage of about 25 mg to about 6,000 mg.

4. The composition of claim 1, wherein the amount effective for treating organ or tissue fibrosis comprises a daily dosage of about 50 mg to about 2000 mg.

5. The composition of claim 1, wherein the amount effective for treating organ or tissue fibrosis comprises a daily dosage of about 100 mg to about 1000 mg.

6. A pharmaceutical composition comprising the composition of claim 1 and a pharmaceutically acceptable carrier.

7. A method of treating organ or tissue fibrosis, comprising administering to a subject an effective amount of a composition comprising one or more 5-methyl-1-(substituted phenyl)-2(1H)-pyridones having a formula of: 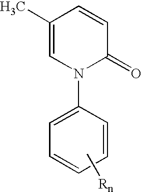
wherein n=1 or 2; R is selected from the group consisting of F, Cl, Br, I, nitro, C₁-C₆ straight-chain alkyl group, C₃-C₆ branched-chain alkyl group, C₁-C₆ straight-chain alkoxy group, C₃-C₆ branched-chain alkoxy group, and halogenated C₁-C₆ alkyl group; and when n=2, not both R are nitro.

8. The method of claim 7, wherein the 5-methyl-1-(substituted phenyl)-2(1H)-pyridone is selected from the group consisting of 1-(2′-bromophenyl)-5-methyl-2(1H)-pyridone, 1-(3′-bromophenyl)-5-methyl-2(1H)-pyridone, 1-(4′-bromophenyl)-5-methyl-2(1H)-pyridone, 1-(2′-chlorophenyl)-5-methyl-2(1H)-pyridone 1-(3′-chlorophenyl)-5-methyl-2(1H)-pyridone, 1-(2′-fluorophenyl)-5-methyl-2(1H)-pyridone, 1-(3′-fluorophenyl)-5-methyl-2(1H)-pyridone, 1-(4′-fluorophenyl)-5-methyl-2(1H)-pyridone, 1-(2′-iodophenyl)-5-methyl-2(1H)-pyridone, 1-(3′-iodophenyl)-5-methyl-2(1H)-pyridone, 1-(4′-iodophenyl)-5-methyl-2(1H)-pyridone, 1-(2′,3′-dibromophenyl)-5-methyl-2(1H)-pyridone, 1-(2′,4′-dibromophenyl)-5-methyl-2(1H)-pyridone, 1-(2′,5′-dibromophenyl)-5-methyl-2(1H)-pyridone, 1-(2′,6′-dibromophenyl)-5-methyl-2(1H)-pyridone, 1-(3′,4′-dibromophenyl)-5-methyl-2(1H)-pyridone, 1-(3′,5′-dibromophenyl)-5-methyl-2(1H)-pyridone, 1-(2′,3′-dichlorophenyl)-5-methyl-2(1H)-pyridone, 1-(2′,4′-dichlorophenyl)-5-methyl-2(1H)-pyridone, 1-(2′,5′-dichlorophenyl)-5-methyl-2(1H)-pyridone, 1-(2′,6′-dichlorophenyl)-5-methyl-2(1H)-pyridone, 1-(3′,4′-dichlorophenyl)-5-methyl-2(1H)-pyridone, 1-(3′,5′-dichlorophenyl)-5-methyl-2(1H)-pyridone, 1-(2′,3′-difluorophenyl)-5-methyl-2(1H)-pyridone, 1-(2′,4′-difluorophenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluorophenyl)-5-methyl-2(1H)-pyridone, 1-(3′,5′-difluorophenyl)-5-methyl-2(1H)-pyridone, 5-methyl-1-(2′-trifluoromethylphenyl)-2(1H)-pyridone, 5-methyl-1-(4′-trifluoromethylphenyl)-2(1H)-pyridone, 1-(2′,3′-bis-trifluoromethylphenyl)-5-methyl-2(1H)-pyridone, 1-(2′,4′-bis-trifluoromethylphenyl)-5-methyl-2(1H)-pyridone, 1-(2′,5′-bis-trifluoromethylphenyl)-5-methyl-2(1H)-pyridone, 1-(2′,6′-bis-trifluoromethylphenyl)-5-methyl-2(1H)-pyridone, 1-(3′,4′-bis-trifluoromethylphenyl)-5-methyl-2(1H)-pyridone, or 1-(3′,5′-bis-trifluoromethylphenyl)-5-methyl-2(1H)-pyridone. 5-methyl-1-(2′-methylphenyl)-2(1H)-pyridone, 1-(3′-methylphenyl)-5-methyl-2(1H)-pyridone, 1-(2′,3′-dimethylphenyl)-5-methyl-2(1H)-pyridone, 1-(2′,4′-dimethylphenyl)-5-methyl-2(1H)-pyridone, 1-(2′,5′-dimethylphenyl)-5-methyl-2(1H)-pyridone, 1-(2′,6′-dimethylphenyl)-5-methyl-2(1H)-pyridone, 1-(3′,4′-dimethylphenyl)-5-methyl-2(1H)-pyridone, 1-(3′,5′-dimethylphenyl)-5-methyl-2(1H)-pyridone, 1-(2′-methoxyphenyl)-5-methyl-2(1H)-pyridone, 1-(3′-methoxyphenyl)-5-methyl-2(1H)-pyridone, 1-(2′,3′-dimethoxyphenyl)-5-methyl-2(1H)-pyridone, 1-(2′,4′-dimethoxyphenyl)-5-methyl-2(1H)-pyridone, 1-(2′,5′-dimethoxyphenyl)-5-methyl-2(1H)-pyridone, 1-(2′,6′-dimethoxyphenyl)-5-methyl-2(1H)-pyridone, 1-(3′,4′-dimethoxyphenyl)-5-methyl-2(1H)-pyridone, and 1-(3′,5′-dimethoxyphenyl)-5-methyl-2(1H)-pyridone.

9. The method of claim 7, wherein the amount effective for treating organ or tissue fibrosis comprises a daily dosage of about 25 mg to about 6,000 mg.

10. The method of claim 7, wherein the amount effective for treating organ or tissue fibrosis comprises a daily dosage of about 50 mg to about 2000 mg.

11. The method of claim 7, wherein the amount effective for treating organ or tissue fibrosis comprises a daily dosage of about 100 mg to about 1000 mg.

12. The method of claim 7, wherein the organ or tissue fibrosis is selected from the group consisting of glomerulus sclerosis, renal interstitial fibrosis, liver fibrosis, pulmonary fibrosis, peridoneal fibrosis, myocardiac fibrosis, fibrosis of skin, post-surgical adhesion, benign prostate hypertrophy, musculoskeletal fibrosis, scleroderma, Alzheimer's disease, fibrotic vascular disease, and glaucoma.

13. The method of claim 7, wherein the composition is administered through a route selected from the group consisting of oral administration, parenteral administration, nasal administration, rectal administration, vaginal administration, ophthalmic application, and topical application.

14. The method of claim 7, wherein the 5-methyl-1-(substituted phenyl)-2(1H)-pyridone is 1-(3′-fluorophenyl)-5-methyl-2(1H)-pyridone.

15. The method of claim 14, wherein the subject experiences less toxicity than treatment with 5-methyl-1-phenyl-2(1H)-pyridone.

16. A compound of 5-methyl-1-(substituted phenyl)-2(1H)-pyridone selected from the group consisting of 1-(2′-chlorophenyl)-5-methyl-2(1H)-pyridone, 1-(3′-chlorophenyl)-5-methyl-2(1H)-pyridone, 1-(2′-fluorophenyl)-5-methyl-2(1H)-pyridone, 1-(3′-fluorophenyl)-5-methyl-2(1H)-pyridone, 1-(4′-fluorophenyl)-5-methyl-2(1H)-pyridone, 1-(2′-iodophenyl)-5-methyl-2(1H)-pyridone, 1-(3′-iodophenyl)-5-methyl-2(1H)-pyridone, 1-(4′-iodophenyl)-5-methyl-2(1H)-pyridone, 1-(2′,3-difluorophenyl)-5-methyl-2(1H)-pyridone, 1-(2′,4′-difluorophenyl)-5-methyl-2(1H)-pyridone, 1-(2′,5′-difluorophenyl)-5-methyl-2(1H)-pyridone, 1-(2′,6′-difluorophenyl)-5-methyl-2(1H)-pyridone, 1-(3′,4′-difluorophenyl)-5-methyl-2(1H)-pyridone, 1-(3′,5′-difluorophenyl)-5-methyl-2(1H)-pyridone and 1-(3′,4′-dichlorophenyl)-5-methyl-2(1H)-pyridone.

17. A pharmaceutical composition comprising one or more of the compound of claim 16 and a pharmaceutically acceptable carrier.

18. A method of treating organ or tissue fibrosis, comprising administering to a subject an effective amount of the composition of claim 17.

19. The method of claim 18, wherein the amount effective for treating organ or tissue fibrosis comprises a daily dosage of about 25 mg to about 6,000 mg.

20. The method of claim 18, wherein the amount effective for treating organ or tissue fibrosis comprises a daily dosage of about 50 mg to about 2000 mg.

21. The method of claim 18, wherein the amount effective for treating organ or tissue fibrosis comprises a daily dosage of about 100 mg to about 1000 mg.

22. The method of claim 18, wherein the organ or tissue fibrosis is selected from the group consisting of glomerulus sclerosis, renal interstitial fibrosis, liver fibrosis, pulmonary fibrosis, peridoneal fibrosis, myocardiac fibrosis, fibrosis of skin, post-surgical adhesion, benign prostate hypertrophy, musculoskeletal fibrosis, scleroderma, Alzheimer's disease, fibrotic vascular disease, and glaucoma.

23. The method of claim 18, wherein the composition is administered through a route selected from the group consisting of oral administration, parenteral administration, nasal administration, rectal administration, vaginal administration, ophthalmic application, and topical application.

24. The method of claim 18, wherein the subject experiences less toxicity than treatment with 5-methyl-1-phenyl-2(1H)-pyridone.
